(19) United States
(12) Patent Application Publication
(10) Pub. No.: US 2025/0129324 A1
(43) Pub. Date: Apr. 24, 2025

(54) LIQUID MICROBIAL GROWTH SYSTEM

(71) Applicant: MARK AND SHAUNA SOUZA GAALSWYK TRUST, Lake Crystal, MN (US)

(72) Inventor: Mark Knute Gaalswyk, Lake Crystal, MN (US)

(21) Appl. No.: 18/493,360

(22) Filed: Oct. 24, 2023

(51) Int. Cl. C12M 1/00; C12M 1/26; C12M 1/34; C12M 1/42

(52) U.S. Cl. CPC C12M 47/02; C12M 23/40; C12M 23/58; C12M 33/14; C12M 35/04; C12M 41/44

(57) ABSTRACT

A liquid microbial growth system includes a frame, a loading system associated with the frame, a shredder adjacent the loading system, and a screw press below the shredder. Positioned below the screw press is a rotating screen filter that delivers a liquid biomass byproduct to a collection tank. A control panel, based on sensed information, determines adjustments needed to the liquid biomass byproduct. The liquid biomass byproduct is then pumped to a pre sterilized production tank and mixed with a PH liquid supply, a Brix sugar supply, a microbial seed supply, and/or freshwater to create the determined adjusted mixture.

### CROSS REFERENCE TO RELATED APPLICATION

This application claims the benefit U.S. Provisional Patent Application Ser. No. 63/380,885 filed Oct. 25, 2022, the contents of these applications are hereby incorporated by reference in their entirety.

### BACKGROUND

This disclosure generally relates to a microbial growth system. More specifically, the disclosure relates to a liquid microbial growth system that pre-sterilizes the tote, or alternatively tank or other container, intakes biomass, shreds the biomass, squeezes liquid from the biomass, screens and analyzes the liquid, pumps the liquid to a tote, injects a live microbial seed into the tote. The resulting combination when applied to a crop reduces the amount of needed fertilizer and watering to a significant and advantageous degree.

Microbial fertilizer, also known as biofertilizer, refers to a substance that contains living microorganisms, such as bacteria, fungi, and algae, which can enhance plant growth by increasing the supply or availability of primary nutrients to the host plant. These microorganisms typically colonize the rhizosphere or the interior of the plant and promote growth by various mechanisms such as nitrogen fixation, solubilizing phosphorus, producing plant hormones, or protecting the plant from pathogens.

These fertilizers are considered a sustainable alternative to chemical fertilizers, as they can improve soil fertility and crop productivity while reducing the negative environmental impact associated with the excessive use of synthetic fertilizers. Additionally, they can help in restoring the natural microbial diversity and balance in the soil, which is crucial for maintaining long-term soil health and productivity. Microbial fertilizers can be applied to seeds, soil, or plant surfaces and are becoming increasingly popular in sustainable agriculture practices due to their ability to improve soil health, promote plant growth, and reduce the dependence on chemical inputs.

Microbial fertilizers are typically produced through a carefully controlled process that involves isolating, culturing, and propagating beneficial microorganisms. One step in the process involves both careful pre-sterilization and then providing the appropriate growth medium and environmental conditions such as temperature, pH, Sugar Brix Level, and carefully controlled aeration, or controlled total lack thereof, to support the rapid multiplication of the microorganisms along with incorporating the microorganisms into a carrier material or a suitable substrate that can protect and preserve the viability of the microorganisms during storage and application. Common carrier materials include derived plant sugars from the liquid of biomass, peat, vermiculite, compost, or other organic materials that provide nutrients and a suitable environment for the microorganisms to grow and multiply. In production of microbial fertilizers careful attention to pre-sterilization of tote or tanks, maintaining the viability and activity of the beneficial microorganisms throughout the manufacturing process, storage and application is important to ensure their effectiveness in promoting plant growth and enhancing soil fertility.

Producing microbial fertilizer can present several challenges that need to be carefully addressed to ensure the viability, effectiveness, and quality of the final product. Some of the difficulties include ensuring the viability and activity of the microorganisms throughout the production process. Factors such as precise pre-sterilization of tote or tank, temperature, pH, oxygen levels, Sugar Brix levels, and nutrient availability must be carefully monitored and controlled to maintain the desired microbial population and growth activity. Any fluctuations or deviations in these conditions can lead to a decrease in the effectiveness of the microbial fertilizer.

Developing a stable formulation that can protect microorganisms during storage and transportation is essential. The chosen carrier material must provide adequate protection against environmental stressors, such as temperature variations, moisture, and UV radiation, which can affect the viability of the microorganisms. Implementing robust quality control measures to ensure the consistency and efficacy of the final product is critical. Current methods are costly and time-consuming. Also, scaling up production to meet the demand for microbial fertilizers can be challenging as increasing production while maintaining the quality and efficacy of the product is also costly and time consuming.

Therefore, an objective of the present disclosure is to provide a modular liquid microbial growth system that is modular, fully automated, provides precise pre-sterilization and reliable.

A further objective of the present disclosure is to provide a modular liquid microbial growth system that is of a standard shipping container size dimension, with standard shipping container corner fastening blocks to provide an easy to transport, easy to start up and much less costly than a traditional “stick built” plant.

Various aspects, features, and advantages of the invention will become apparent from the specification and drawings.

### SUMMARY OF THE INVENTION

A liquid microbial growth system includes a frame having a loading system associated with the frame. An automatic pre-sterilization tote system that utilizes both a caustic solution and also an ultra-violet light in a fully automated sterilization system. A shredder is positioned adjacent the loading system and a screw press is positioned below the shredder. A rotating screen filter is positioned below the screw press and a collection tank receives liquid biomass byproduct from the rotating screen filter. Based on information sensed from the collection tank a control panel determines adjustments needed for the liquid biomass byproduct. Remaining solids are discharged to the edge of modular frame.

The loading system includes a hopper in communication with a conveyor that extends at an upward angle from the hopper to the shredder and has a sensor positioned between the conveyor and the shredder to detect material build up. A second sensor is positioned between the shredder and the screw press to also detect material build up to prevent biomass plug up.

The collection tank has a plurality of sensors to detect an amount of PH and Sugar Brix in the liquid biomass byproduct and to detect the level of the liquid biomass byproduct within the collection tank. A conduit is connected to and in communication with the collection tank to transport the liquid derived from the biomass product to one or more sterilized production tanks disposed within the frame. The production totes or tanks are pre-cleaned and sterilized with a cleaning solution and an infrared blue light that extends into the sterilized production tanks.

When a mid-level is detected in the collection tank a signal is sent to the control panel wherein the control panel begins to analyze the liquid biomass byproduct. Based on information from the PH level sensor and the Brix Sugar sensor the control panel determines the adjustments needed to the liquid biomass byproduct for use as a liquid microbe fertilizer based on a desired soil enrichment and food production. This information is then fed into the software contained in the PLC. The software in the PLC will then automatically adjust simultaneously the speed of the VFDs controlling the motors on the pumps of the biomass liquid collection tank, clean fresh water, one or more of the PH and Sugar Brix solution supply tanks and also the liquid microbial premix such that the amount of liquid flowing through the liquid flow meters, or alternatively loss in weight scales, of each of the totes results in an ideal formulation of derived biomass liquid, of an exact pH, of an exact Sugar Brix Level. Optionally, to also control temperature, the resulting final solution will be fed through a liquid heat exchanger mounted on the module as the liquid flows on its way to one or more of the working tote or tank collectors. When the level of the liquid derived from the biomass byproduct is detected in the working tote or tank as full a signal is sent to the control panel which temporarily stops the operation of all equipment upstream and switches all filling operations to the second working production tote or tank providing such container has already completed its pre-cleaning sterilization cycle. The end result is one working production tote or tank is typically being sterilized and cleaned while the other working production tote or tank is filling with the combination of biomass liquid, Sugar Brix Adjusting Solution, PH adjusting solution, clean freshwater, and liquid seed microbe to obtain an ideal liquid microbial fertilizer solution.

When the collection tank is full the control panel activates pumps that pump the liquid derived from the biomass byproduct from the collection tank, a PH adjusting fluid from a PH adjusting supply tote, Brix Sugar fluid from a Brix adjusting supply tote, microbial seed from a microbial seed tote, and clean freshwater from a clean freshwater supply line to the sterilized production tank based upon the determined adjustments needed. The temperature of the liquid is adjusted by pumping the fluid through a heat exchanger. When the sterilized production tank is full, based on a signal from a liquid level sensor a notification signal is activated by the control panel to alert the operator via a colored light indicator that it is time to remove a production tote or tank and replace with an empty tote or tank.

This has outlined, rather broadly, the features, advantages, solutions, and benefits of the disclosure in order that the description that follows may be better understood. Additional features, advantages, solutions, and benefits of the disclosure will be described in the following. It should be appreciated by those skilled in the art that this disclosure may be readily utilized as a basis for modifying or designing other structures and related operations for carrying out the same purposes of the present disclosure. It should also be realized by those skilled in the art that such equivalent constructions and related operation do not depart from the teachings of the disclosure as set forth in the appended claims. The novel features, together with further objects and advantages, will be better understood from the following description when considered in connection with the accompanying Figures. It is to be expressly understood, however, that each of the Figures is provided for the purpose of illustration and description only and is not intended as a definition of the limits of the present disclosure.

### BRIEF DESCRIPTION OF THE DRAWINGS

FIG. 1 is a perspective view of a liquid microbial system;

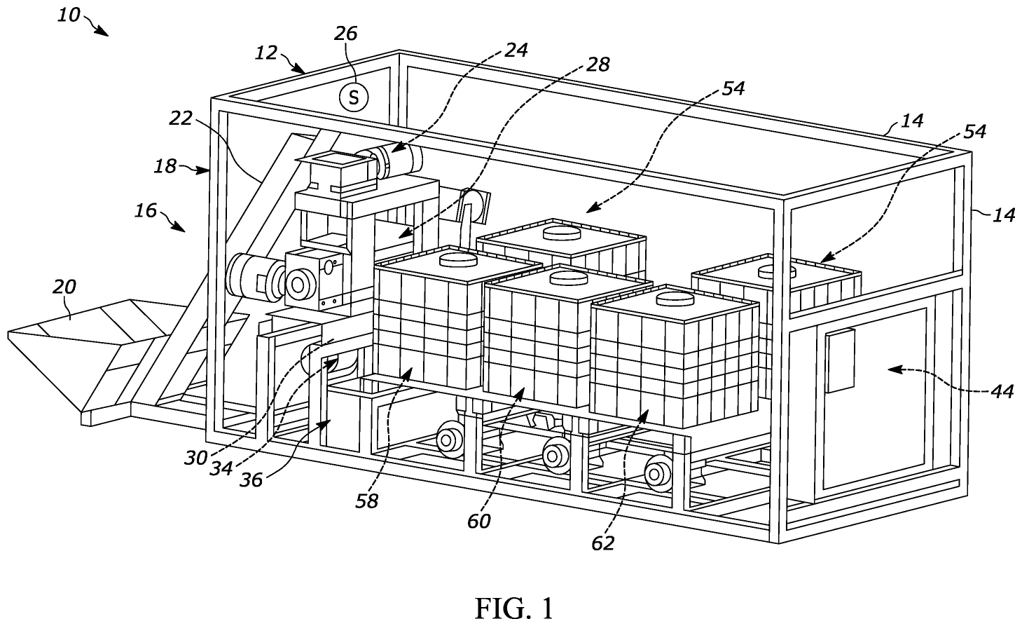

FIG. 2 is a top plan view of a liquid microbial system; and

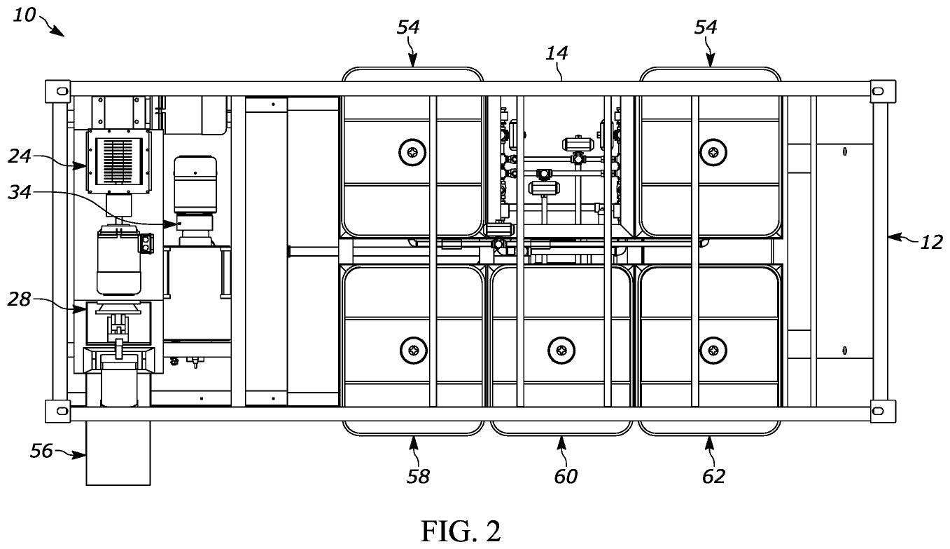

FIG. 3 is a schematic view of the environment of a liquid microbial system.

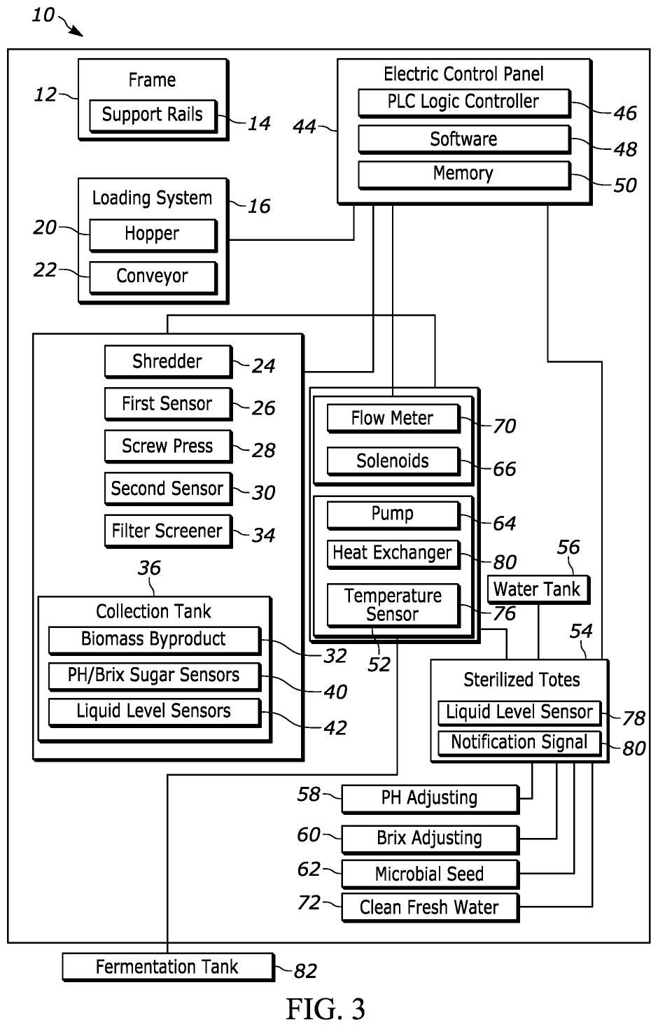

### DETAILED DESCRIPTION

The disclosure described herein is directed to different aspects of a liquid microbial growth system, also referred to as the “unit”. The detailed description set forth below, in connection with the appended drawings, is intended as a description of various configurations and is not intended to represent the only configurations in which the concepts described herein may be practiced. These descriptions include specific details for the purpose of providing a thorough understanding of the various concepts. It will be apparent, however, to those skilled in the art that these concepts may be practiced without these specific details.

Referring to the figures, the liquid microbial growth system 10 includes a frame 12 having a plurality of both vertical and horizontal support rails 14. While the frame 12 can be of any standard shipping container sized frame and shape, in the example shown the frame 12 is the size of a standardized shipping container with connection blocks in each corner for transportation by a standard shipping container transport truck. For example, the frame 12 may either be a standard twenty-foot or forty-foot shipping container size with either standard low height or the high sided shipping container.

The system 10 is a modularized, fully self-contained, fully automatic system that is pre-assembled and pre-configured within the frame 12. At a first end 18 of the frame 12 is a loading system 16 that in one example includes a hopper 20 in communication with a conveyor 22 that extends at an upward angle from the hopper 20 to a shredder 24. Positioned between the conveyor 22 and the shredder 24 is a sensor 26 that detects if there is biomass material build up sufficient to plug the system 10. The shredder 24 is of any type and in one example includes a plurality of knives (not shown) operated by a motor (not shown). The conveyor 22 is operated by a motor such as a variable frequency drive (VFD). The shredder 24 is used to shred any waste food or green biomass material. Positioned below the shredder 24 is a screw press 28. The screw press 28 is of any type but in one example has a plate with a screen preset at a determined restrictive force. Between the shredder 24 and the screw press 28 is a second sensor 30 that detects whether buildup of biomass material is present. The screw press 28 is used to squeeze any liquid (juice) from the waste food or biomass material to create a biomass byproduct 32 where substantially all water is removed.

In one embodiment the system 10 has a filter screener or rotating screen filter 34 used to remove any remaining small pieces of biomass from the biomass byproduct 32. The rotating screen filter is positioned directly below the screw press 28. The remaining liquid biomass byproduct flows to a collection tank 36 while the remaining solid biomass is removed from the system through and exit via a conveyor or the like and is not part of the present invention. A third sensor 38 is positioned between the screw press 28 and the rotating screen filter 34 to detect the level or flow of liquid biomass byproduct 32 feeding into the rotating screen filter 34.

The collection tank 36 has sensors 40 to detect the PH level and the Sugar Brix level of the liquid biomass byproduct 32. Also included in the collection tank 36 is one or more sensors 42 to detect different fluid levels within the collection tank 36. Connected to all sensors and motors is an electric control panel 44 having a PLC logic controller 46, software 48, and memory 50.

Connected to and in communication with the collection tank 36 is a conduit 52 that selectively transports the liquid biomass byproduct 32 to one or more sterilized production totes 54. The one or more sterilized production totes 54 are disposed within the frame 12 of the system 10 and are positioned upon the support rails 14.

The production totes 54 are sterilized prior to production by a cleaning system that automatically, based on a signal from the control panel 44, pumps a cleaning solution into the production tote 54 wherein the cleaning solution is captured in a waste tank 56 for reuse. The cleaning solution is of any type such as a strong caustic or other liquid cleaning solution. An infrared blue light extending into the production tote 54 is also used to further sterilize the production tote 54.

In operation, waste food or green biomass is placed into hopper 20 and transported via conveyor 22 to the shredder 24. If sensor 26 detects a plug or buildup of the waste or biomass material a signal is sent to the control panel 44 which activates and adjusts the speed of the VDF motor of the conveyor so that the rate of incoming biomass material matches the shredding capacity of the shredder 24. The speed of the knives on the shredder are preset by the control panel 44 based on the size of biomass particles desired. The speed is preset higher to obtain finer particles and a lower speed if more coarse particles are desired.

Shredded biomass material then flows to the screw press 28. If second sensor 30 detects buildup between the shredder 24 and the screw press 28 a signal is sent to the control panel which activates the motor of the conveyor 22 to slow down the rate of the incoming waste food or green biomass. The shredded biomass material is pressed by a plate at a preset restrictive force to produce a liquid biomass byproduct 32 from the green biomass or waste food which often is as much of 70% of the incoming weight. The liquid biomass byproduct flows from the screw press 28 to the rotating screen filter 34 where all remaining biomass particles are removed. From the rotating screen filter 34 the liquid biomass byproduct flows to the collection tank 36.

Then at least one liquid sensor 42 in the collection tank 36 will detect different levels of the liquid biomass byproduct 32 in the collection tank 36. When the liquid level in the collection tank 36 is low sensor 42 will send a signal to the control panel 44 and then tank discharge pump is turned off until sensor observes a liquid level at which time tank discharge pump is then turned back on. When the sensor 42 detects a mid-level has been reached within the collection tank 36 a signal is sent to the control panel 44 indicating that the liquid biomass byproduct 32 may be monitored and analyzed and then all liquids will be mixed at a ratio on their way to one of the two working production tanks. Finally, when a high level is detected by sensor 42 a signal is sent to the control panel 44 which momentarily stops all equipment upstream to permit the fluid in the production tote to be removed before the equipment is restarted.

When sensor 42 indicates that a mid-level has been reached within the collection tank additional information received from the PH level and Sugar Brix sensors is used by the control panel 44 to determine the PH level and the Sugar Brix level of the liquid biomass byproduct 32 in the collection tank 36. Once the PH level and the Sugar Brix level of the liquid biomass byproduct 32 is determined the control panel 44 will determine the extent that the liquid biomass byproduct 32 needs to be adjusted for use as the liquid microbe fertilizer with such final formulation stored in the PLC controller based on the desired soil enrichment and food production. This determination may include the amount of liquid from a PH adjusting supply tote 58, from a Brix adjusting supply tote 60, a microbial seed tote 62 containing live inoculant seed microbes, freshwater supply and/or any other liquids needed to optimize the final solution needed to grow the microbe which would include the exact mixture of both microbe seed liquid needed along with the exact amount of liquid derived from the biomass biproduct 32 and any additional liquids needed to optimize the final solution.

Once the mid level of tank 36 is obtained control panel 44 activates a pump 64 to pump fluid from the collection tank 36 to at least one or more sterilized totes 54 while simultaneously also pumping in at precise liquid volumes the needed Sugar Brix adjusting solution, the needed PH adjusting solution, freshwater and liquid microbial seed solutions all together to achieve and ideal formulated microbial growth solution. The speed of the pump 64 is preset and controlled by the control panel 44 but the amount of flow from this pump as determined by the inline liquid flow meter will then direct PLC to mix in the correct proportion of the other liquids to obtain an ideal liquid microbial growth solution. If more than one sterilized tote 54 is used the control panel 44 activates a bank of air solenoids 66 which activate a ball valve 70 to direct the flow of fluid to the desired sterilized tote 54. The liquid biomass byproduct 32 is pumped through a flow meter 70. Simultaneously additional pumps 64 are activated to pump fluid from the PH adjusting supply tote 58, the Brix adjusting supply tote 60, the microbial seed tote 62 and/or a clean freshwater supply 72 to the desired sterilized tank 54 at a rate to provide the exact mixture determined by the control panel 44. As a result, the selected sterilized tote 54 is filled with liquid biomass byproduct 32 from the collection tank at a rate monitored by the flow meter 70 based on an analysis and determination made by the control panel 44. The liquid biomass byproduct 32 is mixed with the PH and Brix adjusting fluids contained in totes 58 and 60 and clean freshwater 72 at a rate determined by the control panel to obtain an ideal mixture that includes the live inoculant seed microbes from tote 62. The temperature of any of the liquids may be adjusted by pumping the fluid through a heat exchanger 74 having a temperature sensor 76 connected to and operated by the control panel 44.

The process continues until the selected sterilized tote 54 is full which is determined by a liquid level sensor 78. Once full a notification signal 80 such as a colored light will notify an operator that the tote 54 is ready to be removed and transferred to external storage for further fermenting, growth, and multiplying of the seed microbe.

In the event the system does not use totes 54 sitting within the shipping container size frame 12 of the unit, it may alternatively pump continuously the exact blend of extracted food or green biomass juice, along with the liquid seed microbe, and either water or some other material simultaneously into fermentation tank 82 external of the modular frame.

The unit shall have a display screen on the control panel for operation, and/or, the ability to connect a remote computer screen for control. The unit will store and be able to transmit all necessary production data for record keeping and billing purposes. The unit will be able to be remotely monitored and parameters adjusted for remote optimizing and repairs. The unit may or may not also have a GPS location positioner device on it so its whereabouts in the world can be determined at all times.

The unit comprises one or more of:
16 tons per 8-hour day shredder capacity
Goal is 498,417 gallons per year of liquid microbe fertilizer production (if 50 percent moisture incoming waste on an 8-hour day) VFD speed control on shredder
Screw press for removing water from incoming waste material
VFD speed control on screw press
Stainless steel solids discharge auger with stainless chute
Power Screener to screen liquid material
VFD on power screener
One variable flow up to 50 GPM Tote fill pump (goal is to cycle a tote every 15 minutes)
Three variable flow up to 10 GPM supply tote pumps.
Two tote batch fill stations
Operator Indicator lights for each fill tote
Each Fill tote has a high-level sensor.
One Cleaning Liquid (CIP) Supply Tote station with level meter.
Two Supply Totes with level meters.
Fully automatic Clean in Place System to clean totes
Screen Reclaim to screen CIP.
Automatic Plant Liquid Characteristic Sensor.
Automatic formulation control based on Plant Liquid characteristics VFD Speed control of all pumps
Three Sight Glasses to monitor actual material flow.
Two Tote Automatic clean in place system with spray balls.
Gallon usage meters on CIP and both supply totes.
Gallon meter on flow line existing bio mass liquid tank 36.
Gallon Fill Meters on both fill totes with totalizer
Tote over fill sensors on both fill totes
Automatic air solenoid valves for all automatic ball valves
Ball valve in position verification on all valves
Automatic powered air control dryer to dry control air to prevent valve misfunction.
Automatic three-way ball valves on each of two batching fill totes
Operator indicator lights for waste filling hopper
Stainless steel green waste fill hopper
Level Sensor up at top of conveyor to auto detect obstruction and slow or stop conveyor
Three Emergency shut off buttons at different locations on module for operator Safety
Large painted control panel with door disconnect and e stop
Cabinet Cooler
Full color HMI computer control screen with color graphics
Fully Automatic plc control system
Remote monitoring capability
Conveyor with Hopper to Convey material up into shredder/press assembly
Five main module support brackets to lift entire module at least 4 inches off floor to provide easy cleaning
Building with Concrete floor (suggest 40×80 or larger) with insulation (
heater for inclement weather operation and racks to store Fermenting totes
230-volt 3-phase power
Air Compressor with piping to module (air dryer included with module)
Used Skid loader and/or forklift for handling waste and 275-gallon totes.
If residual solids not removed with skid loader, conveyor to take away solids

From the above discussion and accompanying figures and claims it will be appreciated that the liquid microbial growth system offers many advantages over the prior art. Although the present disclosure and its advantages have been described in detail, it should be understood that various changes, substitutions, modifications, and alterations can be made herein without departing from the technology of the disclosure as defined by the appended claims. The scope of the present application is not intended to be limited to the particular configurations of the process, machine, manufacture, composition of matter, means, methods and steps described in the specification only expressly stated otherwise. As one of ordinary skill in the art will readily appreciate from the disclosure, processes, machines, manufacture, compositions of matter, means, methods, or steps, presently existing or later to be developed that perform substantially the same function or achieve substantially the same result as the corresponding configurations described herein may be utilized according to the present disclosure. Accordingly, the appended claims are intended to include within their scope such processes, machines, manufacture, compositions of matter, means, methods, or steps.

What is claimed is:

1. A liquid microbial growth system, comprising:
a frame;
a loading system associated with frame;
a shredder adjacent the loading system;
a screw press positioned below the shredder;
a rotating screen filter positioned below the screw press;
a collection tank configured to receive liquid biomass byproduct from the rotating screen filter; and
a control panel which determines adjustments to the liquid biomass byproduct based on information sensed from the collection tank.

2. The system of claim 1 wherein the loading system includes a hopper in communication with a conveyor that extends at an upward angle from the hopper to the shredder and has a sensor positioned between the conveyor and the shredder to detect material build up.

3. The system of claim 1 wherein a sensor is positioned between the shredder and the screw press to detect material build up.

4. The system of claim 1 wherein the collection tank has a plurality of sensors to detect the amount of PH and Sugar Brix in the liquid biomass byproduct and to detect the level of the liquid biomass byproduct within the collection tank.

5. The system of claim 1 wherein a conduit is connected to and in communication with the collection tank that selectively transports the liquid biomass product to one or more sterilized production tanks disposed within the frame.

6. The system of claim 1 wherein the sterilized production tanks are cleaned with a cleaning solution and an infrared blue light that extends into the sterilized production tanks.

7. The system of claim 4 wherein when a mid-level has been detected in the collection tank a signal is sent to the control panel wherein the control panel begins to analyze the liquid biomass byproduct.

8. The system of claim 4 wherein when the level of the liquid biomass byproduct is detected as full a signal is sent to the control panel which stops the operation of all equipment upstream.

9. The system of claim 7 wherein the control panel, based on information from the PH level sensor and Sugar Brix sensors determines the adjustments needed to the liquid biomass byproduct for use as a liquid microbe fertilizer based on a desired soil enrichment and food production.

10. The system of claim 7 wherein the control panel activates pumps that pump the liquid biomass byproduct from the collection tank, a PH adjusting fluid from a PH adjusting supply tote, Brix Sugar fluid from Brix adjusting supply tote, microbial seed from a microbial seed tote, and clean freshwater from a clean freshwater supply to a sterilized production tank based upon the determined adjustments needed.

11. The system of claim 10 wherein the temperature of any liquid is adjusted by pumping the fluid through a heat exchanger.

12. The system of claim 10 wherein the sterilized production tank has a liquid level sensor and a notification signal that is activated when the sterilized production tank is full.
